(19) United States
(12) Patent Application Publication
(10) Pub. No.: US 2021/0322499 A1
(43) Pub. Date: Oct. 21, 2021

(54) MOXIBUSTION COMPOSITION COMPRISING HEMP FLOWER OR BIOMASS AND/OR CANNABIDIOL OIL

(71) Applicants: Julie Lim GREIF, Rancho Palos Verdes, CA (US); Sanghyuck PARK, Pueblo, CO (US)

(72) Inventors: Julie Lim GREIF, Rancho Palos Verdes, CA (US); Sanghyuck PARK, Pueblo, CO (US)

(21) Appl. No.: 16/852,266

(22) Filed: Apr. 17, 2020

(51) Int. Cl. A61K 36/185; A61K 31/05; A61K 36/282

(52) U.S. Cl. CPC A61K 36/185; A61K 36/282; A61K 31/05

(57) ABSTRACT

The present invention relates to a moxibustion composition, and particularly relates to a moxibustion composition comprising hemp biomass and/or cannabidiol oil.

### BACKGROUND

### 1. Field

The present invention relates to a moxibustion composition. Particularly, the present invention relates to a moxibustion composition comprising biomass containing hemp flowers or hemp flowers and leaves, and/or cannabidiol oil.

### 2. Description of the Related Art

Moxibustion or moxa is an ancient Chinese medical therapy that applies burning herbs (such as dried wormwood and ginger) at certain points on the body to stimulate the circulation of blood and qi. Traditionally, moxibustion has been applied to patients using non-moxa or moxa sticks that can be applied directly or indirectly. While direct moxibustion is defined as the application of moxa sticks to designated acupuncture points on the body surface, herb is placed between moxa sticks and acupuncture points for indirect moxibustion.

Based on numerous sources in Chinese literatures, the therapeutic effect of moxibustion is associated with the treatment of chronic symptoms related to “deficiency” and the prevention of human disorders. Moxa can cause physical changes by lowering blood pressure and controlling the circadian rhythm of blood pressure and mental state. Previous studies have demonstrated that moxibustion is effective in treating cervical nerve dizziness, dysmenorrhea, chemotherapy-induced leukopenia and various emergency medical conditions. Efforts have been made to study moxibustion with or without smoke in parallel with other modes and radiation therapy or Taxol therapy using experimental tumor models. Recent studies have shown that moxibustion is effective in treating hypertension and arthritis. Possible mechanisms of action of moxibustion may be related to temperature-related and non-temperature-related factors that cause several sensory stimuli, including smoke effects, herbal effects, and biophysical effects (far infrared rays).

Cannabis sativa, also known as marijuana or hemp, produces secondary metabolites called cannabinoids that are primarily accumulated in the glandular trichome of plants. The two major cannabinoids are cannabidiolic acid (CBDA) and tetrahydrocannabinolic acid (THCA). The two cannabinoids in acid form are decarboxylated and converted to cannabidiol (CBD) and (−)-Δ⁹-trans-tetrahydrocannabinol (Δ⁹-THC), respectively. Cannabidiol and CBDA are the major constituents of nonpsychotropic (hemp-type) C. sativa L. Hemp-type Cannabis produces large amounts of CBDA and can produce up to 40% or more of the cannabinoid extract from whole plants.

According to the Hemp Farming Act of 2018 in the United States, hemp is defined as C. sativa, which produces less than 0.3% tetrahydrocannabinol (THC), and C. sativa, which produces 0.3% or more of THC, is classified as marijuana.

Hemp can be divided into industrial hemp and medicinal hemp according to the content of the main ingredient. If the value of (THC+CBN)/CBD is greater than 1, it is classified as medicinal hemp. If the value is less than 1, it is classified as industrial hemp. As a result of comparison of 177 types of domestic hemp, it was reported that 12.4% belong to industrial hemp and 87.6% belong to medicinal hemp (Choe S et al., The Pharmaceutical Society of Korea. 2011: 264). THC, a major active ingredient of medicinal hemp, has a strong affinity for CB1 receptors and this affinity is a major mechanism of psychotropic activity. In contrast, CBD, a major active ingredient of industrial hemp, has been shown to have beneficial effects such as anti-inflammatory, antiepileptic, antiemetic, and anti-cancer effects in numerous experiments. Further, its potential as a drug has been suggested based on its action to reduce side effects caused by THC (Englund A M et al., Curr Pharmaceut Des., 18(32):4906-14, 2012; Burstein S et al., Bioorgan Med Chem., 23(7):1377-85, 2015).

THC is one of the common phytocannabinoids produced in C. sativa, and includes Δ⁹-THC and Δ⁸-THC, which have psychotropic effects and cause harmful reactions such as excitement and hallucinations.

CBD is also one of the common phytocannabinoids produced in C. sativa, and phytocannabinoids have been widely studied for their therapeutic potential. CBD has been studied for various therapeutic potentials, including anti-inflammatory agents, antioxidants, neuroprotective agents, anticonvulsants, drugs for panic disorder, anti-anxiety agents, antidepressants, analgesics, anti-tumor agents and antipsychotics.

In spite of the therapeutic effects of CBD, there has been no example of developing CBD or industrial hemp with low THC content and high CBD content into a more effective and convenient formulation.

Accordingly, the present inventors have completed the present invention by developing a moxibustion composition comprising CBD or industrial hemp and confirming that the moxibustion composition is an optimal composition for achieving the effect of CBD or industrial hemp in a patient-friendly manner.

The above information described in this background section is only for improving the understanding of the background of the present invention, and therefore may not include information forming prior art that is known to a person of ordinary skill in the art.

### SUMMARY

The object of the present invention is to provide a moxibustion composition comprising hemp biomass and/or cannabidiol oil.

In order to achieve the object above, the present invention provides a moxibustion composition comprising hemp biomass and/or cannabidiol oil.

The moxibustion composition according to the present invention comprises hemp biomass and/or cannabidiol oil, thereby achieving a superior therapeutic effect of hemp or cannabidiol in a variety of diseases in a non-invasive manner.

### BRIEF DESCRIPTION OF THE DRAWINGS

FIG. 1 is a photograph showing the processing of the wormwood in order.

FIG. 2 is a photograph showing the processing of hemp in order.

FIG. 3 is a photograph showing the moxibustion compositions according to the present invention in the form of moxa cone for direct moxibustion.

FIG. 4 is a photograph showing the moxibustion compositions according to the present invention for smokeless moxibustion.

FIG. 5 is a photograph showing the moxibustion compositions according to the present invention in the form of a stick.

FIG. 6 is a photograph showing the moxibustion composition according to the present invention in the form of moxa cone applied in the moxa bucket moxibustion.

### DETAILED DESCRIPTION

Unless defined otherwise, all technical and scientific terms used herein have the same meaning as commonly understood by a person of ordinary skill in the art to which the present invention belongs. In general, the nomenclature used herein is well known and commonly used in the art.

In the present invention, biomass comprising hemp flowers or hemp flowers and leaves and/or cannabidiol oil is intended to be used in a moxibustion composition.

In one aspect, the present invention relates to a moxibustion composition comprising hemp biomass and/or cannabidiol oil.

In the present invention, the term “biomass” refers to the whole constituents of a plant that synthesizes organic matter by receiving solar energy, specifically, hemp.

In the present invention, the hemp is preferably industrial hemp, and the industrial hemp biomass may be characterized by containing less than 0.3% by weight of tetrahydrocannabinol (THC) based on the total weight of the biomass.

The present invention may be characterized in that the industrial hemp biomass is the biomass of the flowers, or the flowers and leaves of the industrial hemp, or the biomass of the flowers and leaves subjected to a certain processing, for example, peeling process, but is not limited thereto.

In the present invention, the cannabidiol oil may be derived from industrial hemp extract, but is not limited thereto.

In the present invention, the term “extract” is meant to include all materials obtained by extracting the components contained in the natural product, regardless of the extraction method, extraction solvent, extracted component or form of the extract. Additionally the term is interpreted in a broad sense to include all materials that can be obtained by processing or treating by other methods materials obtained by extracting components contained in the natural product.

In one specific example, the processing or treatment may be an additional fermentation, enzymatic treatment, centrifugation, filtration to remove impurities, concentration, or drying of the extract.

Therefore, in the present invention, “extract” is a broad concept including fermentation products, concentrates and dried products. Specifically, the extract in the present specification may be an extract in the form of a liquid, and it is preferably a concentrate of the extract (liquid concentrate), but is not limited thereto. In addition, since the industrial hemp extract in the present invention is preferably in liquid form, the liquid extract and the extract in the present invention are used in substantially the same sense.

In the present invention, the industrial hemp extract may be characterized in that it is extracted with an organic solvent selected from the group consisting of water, organic solvents and mixtures thereof. The organic solvent may be ethanol, coconut oil, hemp seed oil, MCT oil or olive oil, but is not limited thereto.

The extract may be obtained by juice extraction, water vapor extraction, hot water extraction, ultrasonic extraction, solvent extraction, CO₂ extraction or reflux cooling extraction. The solvent extraction may be characterized by using water, lower alcohols of C₁˜C₆, coconut oil, hemp seed oil, medium-chain triglyceride (MCT) oil, olive oil or a mixture thereof, but is not limited thereto. The extract may be the one extracted according to a variety of methods used in the conventional method for producing a natural extract. Preferably, the extract may be the one obtained by CO₂ extraction and/or solvent extraction, more specifically CO₂ extraction and/or ethanol extraction.

In the present invention, CBD oils include, but are not limited to, isolates comprising only pure CBD component, broad spectrum CBD except for tetrahydrocannabinol (THC) in hemp, and full spectrum CBD.

The present invention may be characterized in that the moxibustion composition further comprises herbal materials.

In the present invention, the term “herb” refers to a total of a plant of which leaves, stems, roots, and the like are used for edible and medicinal purposes, or fragrance or flavor is used.

In the present invention, the herbal material may be wormwood, ocher, charcoal, mulberry branches, peach tree, mustard seed, nasturtium or a mixture thereof, but is not limited thereto.

In the present invention, the wormwood may be the wormwood (leaves of wormwood), Artemisia capillaris (Injinho), Iinjin wormwood, Ascoparia, Artemisia princeps Panpan. cv. ssajuari, Kangwha Mani mountain wormwood, Gilsang mountain wormwood, or Haemyeong mountain wormwood, but is not limited thereto.

The present invention may be characterized in that the moxibustion composition comprises the hemp biomass and the herbal materials in a weight ratio of 1:9 to 9:1, preferably 4:6 to 6:4, and more preferably 5:5, but is not limited thereto.

The present invention may be characterized in that the moxibustion composition comprises cannabidiol oil and herbal materials, but the content of the cannabidiol oil is less than 2% by weight of the total composition.

The present invention may be characterized in that the moxibustion composition comprises 0.1 to 10 parts by weight of cannabidiol oil, and 50 to 150 parts by weight of the herbal materials with respect to 100 parts by weight of the hemp biomass. Preferably, the composition may comprise 1 to 5 parts by weight of cannabidiol oil and 70 to 130 parts by weight of the herbal materials, but is not limited thereto.

In the present invention, the moxibustion composition may be formulated into a formulation selected from the group consisting of powder, pill, granule, tablet, capsule, moxa cone, moxa roll (rod shape), and stick type, but is not limited thereto.

In the present invention, the moxibustion composition may be used with moxa bucket moxibustion, but is not limited thereto.

Hereinafter, the present invention will be described in more detail with reference to Examples. These examples are only for illustrating the present invention, and it will be apparent to a person of ordinary skill in the art that the scope of the present invention is not to be construed as limited by these examples.

### Example 1. Preparation of Moxibustion Composition

### Example 1-1. Processing of Industrial Hemp Biomass and Wormwood for Moxibustion Composition

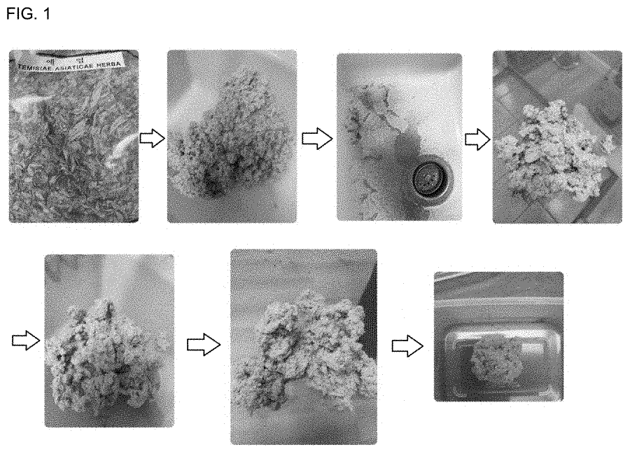

Dried wormwood (dried leaves of wormwood), which has been harvested more than three years ago, was pulverized in a mixer, and then sieved to filter debris and powder. The process was repeated until no debris was left and it became fibers to prepare cotton ball form (FIG. 1).

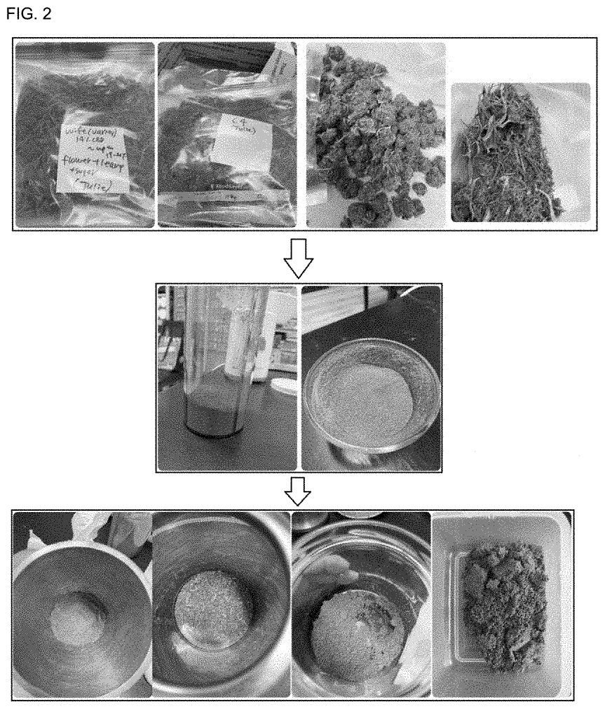

Used hemp were C4 strain (shucked flower and leaves) (Sacred Giftz), blend cheery wine (T1 flower and leaves) (Sacred Giftz), wife strain (shucked flower and leaves) (Sacred Giftz), trimmed flower T1 (Sacred Giftz). Each of the fresh hemp, which has been harvested less than 1-3 months ago, was fully dried, pulverized in a mixer, and then sieved to remove impurities to prepare hemp biomass (FIG. 2).

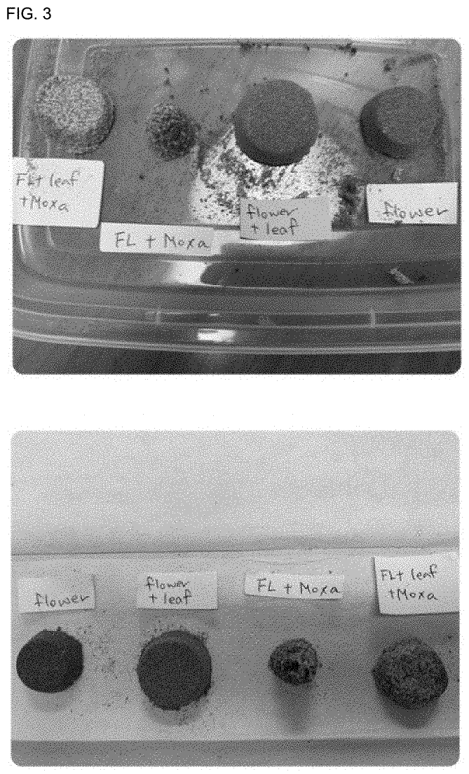

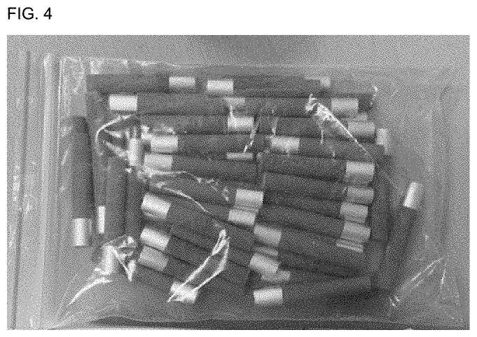

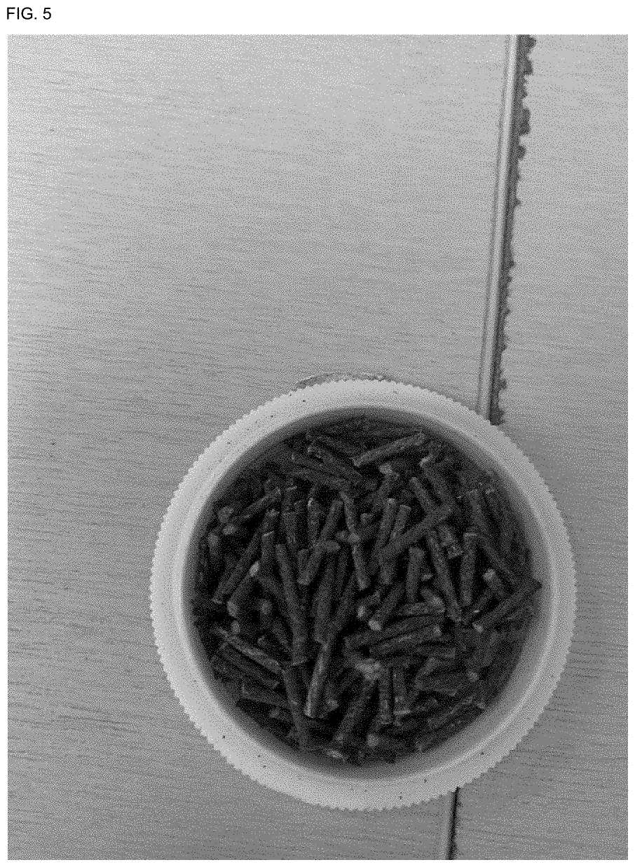

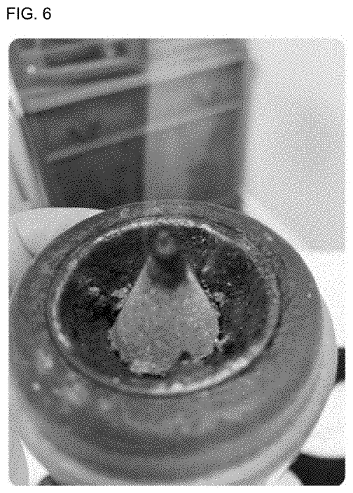

Moxibustion compositions were prepared by blending the processed wormwood and each hemp biomass. A mixture composition of hemp leaves biomass and the processed wormwood, a mixture composition of hemp flowers biomass and the processed wormwood, and a mixture composition of hemp leaves, flowers biomass and the processed wormwood were prepared in the forms for direct moxibustion or indirect moxibustion (FIGS. 3 to 6).

### Example 1-2. Moxibustion Composition Comprising Industrial Hemp Biomass and Wormwood

15 g of the properly pulverized industrial hemp biomass powder was evenly spread on the bottom surface of the cavity formed in the lower mold of the molding machine. 30 g of the properly pulverized wormwood powder was placed on the top of the hemp biomass powder. And 15 g of the industrial hemp biomass powder was evenly placed on the top of the wormwood powder again. Heat was applied up to a temperature of about 130-170° C. and then the upper mold with the core formed was pressed to form a moxibustion composition.

### Example 1-3. Moxibustion Composition Comprising Cannabidiol Solution and Wormwood

A moxibustion composition was prepared by spraying 2 ml of cannabidiol solution to 100 g of wormwood powder and drying the mixture. Spraying and drying were repeated seven times. Cannabidiol oil was obtained by CO₂ extraction of industrial hemp seeds and stems.

### Example 1-4. Moxibustion Composition Comprising Industrial Hemp Biomass, Cannabidiol Solution and Wormwood

200 g of properly pulverized wormwood powder was put into a cavity formed in the lower mold of the molding machine, and heated to a temperature of about 130-170° C. The upper mold with the core formed was pressed to form a moxibustion composition. Then, a mixture of 200 g of properly pulverized industrial hemp biomass powder and 2 ml of cannabidiol solution was applied to the top and bottom of the composition in a thickness of one to several mm to form moxibustion composition. Cannabidiol oil was obtained by CO₂ extraction of industrial hemp seeds and stems.

### Example 2. Confirmation of the Effects of the Moxibustion Compositions According to the Present Invention

The moxibustion compositions prepared in Example 1 were applied to patients with insomnia, anxiety or pain to confirm the therapeutic effects for insomnia, anxiety or pain. In addition, the effects of the moxibustion compositions prepared in Example 1 and general moxa for insomnia, anxiety and pain were compared.

The results of the experiments are shown in the tables below.

| | | | |
|---|---|---|---|
| Test Number | LIM2 | Test Subject | LIM |

| | |
|---|---|
| Test Date | 2020 Feb. 3 |
| Test Theme | Therapeutic effect of CBD moxibustion for pain (finger arthritis) |
| Study Contents | Confirmation of the therapeutic effect of CBD moxibustion on pain, |
| (Objective) | swelling and redness in the middle of the first node of the left thumb |
| Tools and | Moxibustion composition prepared by the method of Example 1 |
| Materials | comprising hemp C4 Strain leaf and flower mixture and wormwood, |
| | incense, lighter, Jaungo, alcohol, cotton, tissue, incense holder, |
| | moxa, two cork board |
| Used | 5 jang* of moxibustion composition were applied to Ashi point. |
| Acupuncture | |
| Point | |
| Results | 1. One treatment relieved pain and smoothed finger movements. |
| | 2. NUT became small. |
| Etc. | One treatment alleviated arthritis. |
| | Arthritis disappeard completely. |

*jang is a unit of moxibustion composition and 1 jang generally may correspond to a size of a rice grain

| | | | |
|---|---|---|---|
| Test Number | Woods 1 | Test Subject | WOODS |

| | |
|---|---|
| Test Date | 2020 Jan. 31 |
| Test Theme | Therapeutic effect of CBD moxibustion composition for insomnia |
| | and anxiety |
| Study Contents | Confirmation of the therapeutic effect of CBD moxibustion at |
| (Objective) | Anmyeon acupuncture point and 3^{rd} toe behind acupuncture point |
| | for insomnia and anxiety |
| Tools and | Moxibustion composition prepared by the method of Example 1 |
| Materials | comprising hemp C4 Strain leaf and flower mixture and wormwood, |
| | incense, lighter, Jaungo, alcohol, cotton, tissue, incense holder, |
| | moxa, two cork board |
| Used | Anmyeon acupuncture point and 3^{rd} toe behind acupuncture point |
| Acupuncture | |
| Point | |
| Results | At 1^{st} jang of moxibustion composition, the patient could feel heat |
| | around moxibustion. After 3^{rd} jang of moxibustion composition, the |
| | patient could feel heat all over the body. |

| | | | |
|---|---|---|---|
| Test Number | Woods2 | Test Subject | WOODS |

| | |
|---|---|
| Test Date | 2020 Feb. 3, 2020 Feb. 7, 2020 Feb. 10, 2020 Feb. 13 |
| Test Theme | Therapeutic effect of CBD moxibustion for insomnia and anxiety |
| | Therapeutic effect of CBD moxibustion for neck pain |
| Study Contents | Confirmation of the therapeutic effect of CBD moxibustion at |
| (Objective) | Anmyeon acupuncture point and 3^{rd} toe behind acupuncture point |
| | for insomnia and anxiety |
| Tools and | Moxibustion composition prepared by the method of Example 1 |
| Materials | comprising hemp C4 Strain leaf and flower mixture (50%) and |
| | wormwood (50%), incense, lighter, Jaungo, alcohol, cotton, tissue, |
| | incense holder, moxa, two cork board |
| Used | Anmyeon acupuncture point and 3^{rd} toe behind acupuncture point |
| Acupuncture | LI10(Susamni) 2 point (both sides) |
| Point | |
| Results | After last treatment, patient's anxiety got better(8/10 → 3/10). |
| | After LI10 moxibustion, neck pain was relieved. |
| Etc. | Follow up: On Feb 7 and Feb 10, anxiety got much better (2-3/10). |
| | On Feb 13, anxiety was zero and the patient felt so much better. |
| | This patient had a 90% reduction in the regular dosage of pain |
| | medication. |
| | Sleep has been improved. |
| | CBD moxibustion was effective. |

| | | | |
|---|---|---|---|
| Test Number | Jung 1 | Test Subject | Jung |

| | |
|---|---|
| Test Date | 2019 Nov. 13 |
| Test Theme | Comparison between general moxibustion (moxa) and CBD |
| | moxibustion (thread moxa) |
| Study Contents | Confirmation and comparison of patient's response (knee pain and |
| (Objective) | golf elbow pain) for general moxibustion and CBD moxibustion |
| Tools and | Moxibustion composition prepared by the method of Example 1 |
| Materials | comprising C4 strain or trimmed flower T1, general moxa kit, |
| | alcohol, cotton |
| Used | 1. Right ST36 (Joksamni) - 9 jang of general moxa |
| Acupuncture | 2. Left ST36 (Joksamni) - 7 jang of CBD L1 moxibustion composition |
| Point | 3. Right LI10 (Susamni) - 5 jang of L1 moxibustion composition |
| | 4. Left LI10 (Susamni) - 5 jang of F1 moxibustion composition |
| | 5. REN4 (Gwanwon) - 7 jang of F1 moxibustion composition |
| Result and | 1. R Side ST36: There was no feeling until 3^{rd} jang of moxa. There |
| supplement | was tingling feeling to tiptoe from 6^{th} jang of moxa. |
| | 2. L Side ST36: Response started from 3^{rd} jang of moxibustion |
| | composition. Patient felt better from 5^{th} jang of moxibustion |
| | composition, but response was still weak at 5^{th} jang of moxibustion |
| | composition. |
| | 3. R Side LI10: Patient felt stimulus to tiptoe from 2^{nd} jang of |
| | moxibustion composition. |
| | 4. L Side LI10: Big 1^{st} jang of moxibustion composition was applied |
| | and patient felt nice. Patient felt stimulus to tiptoe at 2^{nd} jang of |
| | moxibustion composition. |
| | 5. REN4: Patient felt energy at 1^{st} jang of moxibustion composition |
| | and felt nice. Patient felt prick at 2^{nd} jang of moxibustion |
| | composition, had a feeling of energy going on and felt relaxed. |
| | Muscle stiffness disappeared after moxibustion treatment and ROM |
| | got better. Patient felt lighter overall. Knee pain 1/10, elbow pain |
| | 0/10 |
| Conclusion | This patient who had applied general moxa at home felt that the |
| | present moxibustion composition was quite different from the |
| | general moxa and was more intense. After moxibustion treatment, |
| | the patient confirmed effectiveness at full stretching and golf swing |
| | test and felt no pain at all. |

| | | | |
|---|---|---|---|
| Test Number | AHN1 | Test Subject | AHN |

| | |
|---|---|
| Test Date | 2019 Nov. 8 |
| Test Theme | Comparison between general moxibustion (moxa) and CBD |
| | moxibustion |
| Study Contents | Comparison of heat and response (stimulation) rates by applying |
| (Objective) | each moxibustion composition on both legs |
| Tools and | General moxa, Moxibustion composition prepared by the method of |
| Materials | Example 1, alcohol, cotton |
| Used | Right side: SP6 (Samumgyo), SP9 (Umreungcheon) - CBD |
| Meridian | moxibustion composition |
| Point | Left side: SP6 (Samumgyo), SP9 (Umreungcheon) - general moxa |
| Result | After 10 jang of CBD moxibustion composition |
| | After 20 jang of general moxa |
| | Patient felt warmth on legs and felt light and nice (relaxed feeling) |
| | when walking. |
| | CBD moxibustion made feel heat on ankle and knee from 1^{st} jang of |
| | moxibustion composition. |
| | General moxibustion made feel heat on ankle and knee from 16^{th} |
| | jang of moxa. After moxibustion, swelling (edema) from the ankle |
| | to the knee was greatly reduced. |
| Etc. | Patient usually has felt both cold and ache on both legs (from ankle |
| | to calf). He has felt colder in the right instep and leg. |

Through the above experiment, it was confirmed that the moxibustion composition of the present invention exhibits a therapeutic effect of insomnia, anxiety or pain, and the pain relief and response (stimulation) rate are faster than the general moxibustion.

As described above in detail specific parts of the present invention, it will be apparent to a person of ordinary skill in the art that these specific descriptions are merely preferred embodiments, and thus the scope of the present invention is not limited thereto. Thus, the substantial scope of the present invention will be defined by the appended claims and their equivalents.

What is claimed is:

1. A moxibustion composition comprising hemp biomass and/or cannabidiol oil.

2. The moxibustion composition of claim 1, wherein the hemp biomass is industrial hemp biomass.

3. The moxibustion composition of claim 2, wherein the industrial hemp biomass contains less than 0.3% by weight of tetrahydrocannabinol (THC) based on the total weight of the biomass.

4. The moxibustion composition of claim 2, wherein the industrial hemp biomass is the biomass of the industrial hemp flowers or the hemp flowers and leaves.

5. The moxibustion composition of claim 1, wherein the cannabidiol oil is derived from industrial hemp extract.

6. The moxibustion composition of claim 5, wherein the industrial hemp extract is obtained by extraction using the solvent selected from the group consisting of water, organic solvents and mixtures thereof.

7. The moxibustion composition of claim 6, wherein the organic solvent is lower alcohols of C₁˜C₆, coconut oil, hemp seed oil, medium-chain triglyceride (MCT) oil, or olive oil.

8. The moxibustion composition of claim 1, further comprising herbal materials.

9. The moxibustion composition of claim 8, wherein the herbal material is wormwood, ocher, charcoal, mulberry branches, peach tree, mustard seed, nasturtium or a mixture thereof.

10. The moxibustion composition of claim 9, wherein the wormwood is wormwood (leaves of wormwood), Artemisia capillaris (Injinho), Iinjin wormwood, Ascoparia, Artemisia princeps Panpan. cv. ssajuari, Kangwha Mani mountain wormwood, Gilsang mountain wormwood, or Haemyeong mountain wormwood.

11. The moxibustion composition of claim 8, comprising the hemp biomass and the herbal materials in a weight ratio of 1:9 to 9:1.

12. The moxibustion composition of claim 8, wherein the composition comprises cannabidiol oil and herbal materials provided that the content of the cannabidiol oil is less than 2% by weight of the total composition.

13. The moxibustion composition of claim 8, wherein the composition comprises 0.1 to 10 parts by weight of cannabidiol oil and 50 to 150 parts by weight of the herbal materials with respect to 100 parts by weight of the hemp biomass.

14. The moxibustion composition of claim 1, wherein the composition is formulated into a formulation selected from the group consisting of powder, pill, granule, tablet, capsule, moxa cone, moxa roll (rod shape), and stick type.

15. The moxibustion composition of claim 1, wherein the composition is used with moxa bucket moxibustion.
